(19) United States
(12) Patent Application Publication
(10) Pub. No.: US 2016/0158125 A1
(43) Pub. Date: Jun. 9, 2016

(54) PRODUCTS FOR OXIDATIVELY CHANGING THE COLOR OF KERATIN FIBERS, IN A DISPENSER

(71) Applicant: Henkel AG & Co. KGaA, Duesseldorf (DE)

(72) Inventors: Constanze Neuba, Grevenbroich (DE); Norbert Schettiger, Hilden (DE); Burkhard Mueller, Duesseldorf (DE)

(21) Appl. No.: 15/045,323

(22) Filed: Feb. 17, 2016

Related U.S. Application Data
(63) Continuation of application No. PCT/DE2014/200422, filed on Aug. 22, 2014.

(30) Foreign Application Priority Data
Aug. 27, 2013 (DE) 10 2013 217 025.4

(51) Int. Cl. A61K 8/22; A61K 8/46; A61K 8/34; A61K 8/41; A61Q 5/08; A61Q 5/10

(52) U.S. Cl. CPC A61K 8/22; A61Q 5/08; A61Q 5/10; A61K 8/347; A61K 8/415; A61K 8/463; A61K 2800/882; A61K 2800/87

(57) ABSTRACT

A product for oxidatively coloring and/or lightening keratinic fibers includes (1) a dispenser, which has two chambers (A) and (B) separate from one another; an outlet opening (C) communicating with chamber (A) and with chamber (B); (2) a preparation (a) in chamber (A), which preparation includes one or more thickening polymers in a total amount by weight (G1) of 0.1 to 20%, based on preparation (a); one or more fatty components in a total amount by weight (G2) of 0.1 to 20%, based on preparation (a); and (3) a preparation (b) in chamber (B), which preparation includes one or more thickening polymers in a total amount by weight (G3) of 0.1 to 20%, based on preparation (b), and one or more fatty components in a total amount by weight (G4) of 0.1 to 20%, based on the total weight of the preparation (b). The weight ratio of G1/G3 has a value of 0.5 to 2.0 and the weight ratio of G2/G4 has a value of 0.5 to 2.0.

### FIELD OF THE INVENTION

The present invention generally relates to a product for the coloring and/or lightening of keratinic fibers, which product includes a dispenser having two chambers separate from one another, which have a common outlet opening, whereby each of the two chambers in each case includes a preparation with special thickening polymers and special fatty components. Furthermore, the present invention relates to a method in which keratinic fibers are colored and/or lightened upon use of the product.

### BACKGROUND OF THE INVENTION

The changing of the color of keratinic fibers, particularly of hair, is an important field in modern cosmetics. As a result, the appearance of the hair can be adapted both to current fashion trends and to the individual wishes of the individual person. The skilled artisan is aware of various options for changing the color of hair.

Hair color can be changed temporarily by the use of direct dyes. In this case, already formed dyes diffuse from the coloring agent into the hair fiber. Dyeing with direct dyes is associated with less hair damage, but a disadvantage is the low durability and the rapid washing out of the colors obtained with direct dyes.

Oxidative color-changing agents are usually used if the consumer wants a long-lasting color result or a shade that is lighter than the consumer's original hair color. So-called oxidation coloring agents are used for permanent, intensive colors with suitable fastness properties. Such coloring agents typically include oxidation dye precursors, so-called developer components and coupler components, which together form the actual dyes under the influence of oxidizing agents, usually hydrogen peroxide. Oxidation coloring agents are characterized by excellent, long-lasting color results.

The mere lightening or bleaching of hair often occurs with the use of oxidizing agents without the addition of oxidation dye precursors. The use of hydrogen peroxide alone as an oxidizing agent is sufficient for an average bleaching effect; a mixture of hydrogen peroxide and peroxydisulfate salts is generally employed to achieve a greater bleaching effect.

Oxidative color-changing agents are typically found on the market in the form of two-component agents, in which the two components are packaged separately in two separate containers and must be mixed together shortly before use.

The first component is usually a formulation, which has been made alkaline and is often available in the form of a cream or a gel and which, provided that a change in color as well is desired concurrently with the lightening, also includes in addition oxidation dye precursors. This first component is provided in most cases in a tube, less often in a plastic or glass container.

The second component is a formulation, which has usually been made acidic for reasons of stability and which includes hydrogen peroxide in concentrations of 3 to 12% by weight as the oxidizing agent. The oxidizing agent formulation mostly has the form of an emulsion or dispersion and as a rule is made available in a plastic bottle with a reclosable outlet opening (developer bottle).

To prepare the ready-to-use mixture, the consumer must mix the two components together shortly before use. For this purpose, normally the alkaline cream or gel component must be transferred completely from the tube or glass or plastic container into the developer bottle; then both components are mixed together as completely and homogeneously as possible by shaking, and they are finally removed via an outlet opening in the top of the developer bottle.

This separate mixing operation has a number of drawbacks for consumers, however. Thus, the quantity ratio of the two components can be changed by the incomplete emptying of the tube; this results in variations in the desired color result. If the shaking or mixing of the two components is too short, the application mixture is not homogeneous with the result of a nonuniform color result. Moreover, it is also desirable for reasons of ease of use to eliminate this mixing step entirely.

To avoid these drawbacks, multi-chamber containers with a common discharge opening were developed, in which the two components are mixed in the valve or dispenser during the discharging. The removing of the application mixture via the dispenser makes the mixing of the components by the consumer superfluous and has greatly increased the ease of use.

But with these dispensers as well, there still is the problem that the mixing ratio of the two components can change depending on the filling level of the formulations in the chambers, the flow behavior of the two formulations, and depending on the pressure within the dispenser. This poses the great risk that the composition of the application mixture changes during its removal. The associated variations in the color result are very greatly not desired by the consumer.

It has not been possible so far to provide oxidative hair color changing products that are based on a multi-chamber system and enable dosing of the application mixture with a constant, defined composition. The attempt was made in DE 10 2007 056 935 A1 to assure the constancy of the mixing ratio by developing dispensers with a special control mechanism. This mechanism, however, does not rule out incorrect operation on the part of the consumer.

It is therefore desirable to provide a new product for the oxidative coloring and/or lightening of keratinic fibers, which product is based on a dispenser that enables the removal of the finished application mixture. In this case, the application mixture removed via the dispenser should be defined and constant with respect to its composition and not change as a function of the degree of dispenser filling.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A product for the oxidative coloring and/or lightening of keratinic fibers includes a dispenser, which has two chambers (A) and (B) separate from one another, and has an outlet opening (C), which is in communication with chamber (A) and with chamber (B); a preparation (a) in chamber (A), including in a cosmetic carrier one or more thickening polymers in a total amount by weight (G1) of 0.1 to 20% by weight, based on the total weight of preparation (a), one or more fatty components in a total amount by weight (G2) of 0.1 to 20% by weight, based on the total weight of preparation (a); and a preparation (b) in chamber (B), including in a cosmetic carrier one or more thickening polymers in a total amount by weight (G3) of 0.1 to 20% by weight, based on the total weight of preparation (b), one or more fatty components in a total amount by weight (G4) of 0.1 to 20% by weight, based on the total weight of preparation (b); wherein the weight ratio of G1/G3 has a value of 0.5 to 2.0 and the weight ratio of G2/G4 has a value of 0.5 to 2.0.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it was now found that it is possible to obtain an application mixture of invariable composition from a multi-chamber dispenser, if the two preparations in the multi-chamber dispenser are precisely matched with respect to their content and composition of thickening polymers and fatty substances.

A first subject matter of the invention is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b), wherein
the weight ratio G1/G3 has a value of 0.5 to 2.0 and
the weight ratio G2/G4 has a value of 0.5 to 2.0.

Keratinic fibers, keratin-including fibers, or keratin fibers are to be understood to mean pelts, wool, feathers, and in particular human hair. Although the agents of the invention are primarily suitable for lightening and coloring keratin fibers, in principle nothing precludes use in other fields as well.

Preparations (a) and (b) located in chambers (A) and (B) include the essential ingredients in each case in a cosmetic carrier, preferably in a suitable aqueous, alcoholic, or aqueous-alcoholic carrier. For the purpose of oxidative color changing, such carriers can be, for example, creams, emulsions, gels, or surfactant-including foaming solutions as well, such as, for example, shampoos, foam aerosols, foam formulations, or other preparations suitable for use on hair. Preparations (a) and/or (b) are especially preferably creams or emulsions.

A distinguishing feature, essential to the invention, for preparations (a) and (b) is their precisely mutually matched content of thickening polymers. “Thickening polymers” within the meaning of the present invention are understood to mean organic polymeric compounds that increase the viscosity of the water, if they are added in an amount of 0.1% by weight to water (at 22° C. and a pressure of 760 mm Hg). The viscosity is measured in this case at 22° C. (22° C./Brookfield viscometer/spindle 1/10 rpm).

Polymeric compounds within the meaning of the present invention are understood to mean compounds, based on repeat units, with a molar weight of more than 5000 g/mol, preferably of more than 10,000 g/mol, and particularly preferably of more than 20,000 g/mol. The maximum molar weight of the polymers depends on the synthesis conditions, batch size, and the degree of polymerization and preferably does not exceed 10⁸ g/mol.

Compounds based on repeat units have at least 50 repeat units, preferably at least 100 repeat units, and particularly preferably at least 500 repeat units.

Preparation (a), which is located in chamber (A) of the dispenser, includes in a cosmetic carrier one or more thickening polymers in a total amount by weight (G1) of 0.1 to 20% by weight, based on the total weight of preparation (a). Preparation (b), which is located in chamber (B) of the dispenser, includes in a cosmetic carrier one or more thickening polymers in a total amount by weight (G3) of 0.1 to 20% by weight, based on the total weight of preparation (b). In this respect, a feature, essential to the invention, of the product is that the weight ratio G1/G3 has a value of 0.5 to 2.0.

The total amount by weight G1 is understood to mean the total weight of all thickening polymers included in formulation (a). The calculation basis in this case is the total weight of preparation (a).

The total amount by weight G3 is understood to mean the total weight of all thickening polymers included in formulation (b). The calculation basis in this case is the total weight of preparation (b).

If the weight ratio G1/G3 of the thickening polymers in formulations (a) and (b) is within the value range of 0.5 to 2.0, both formulations can be dispensed reproducibly and constantly and via outlet opening of dispenser (C); i.e., it is possible to remove defined and constant amounts of formulations (a) and (b) over the entire application period. An application mixture, prepared from (a) and (b) and having the same composition in each removal step, can be removed in this way via the dispenser, regardless of whether the dispenser is still completely filled or has already been partially emptied.

A very especially preferred product for the oxidative coloring and/or lightening of keratinic fibers is therefore characterized in that the weight ratio of G1/G3 has a value of 0.6 to 1.8, preferably of 0.65 to 1.6, and particularly preferably of 0.7 to 1.4.

Furthermore, their precisely matched content of fatty components is characteristic for preparations (a) and (b). “Fatty components” within the meaning of the invention are understood to mean organic compounds with a solubility in water at room temperature (22° C.) and atmospheric pressure (760 mm Hg) of less than 1% by weight, preferably of less than 0.1% by weight.

The definition of fatty components includes explicitly only uncharged (i.e., nonionic) compounds. Fatty components have at least one saturated or unsaturated alkyl group having at least 8 C atoms. The molar weight of the fatty components is a maximum of 5000 g/mol, preferably a maximum of 2500 g/mol, and particularly preferably a maximum of 1000 g/mol. The fatty components are neither polyoxyalkylated nor polyglycerylated compounds.

Preparation (a), which is located in chamber (A) of the dispenser, includes in a cosmetic carrier one or more fatty components in a total amount by weight (G2) of 0.1 to 20% by weight, based on the total weight of preparation (a). Preparation (b), which is located in chamber (B) of the dispenser, includes in a cosmetic carrier one or more fatty components in a total amount by weight (G4) of 0.1 to 20% by weight, based on the total weight of preparation (b). In this respect, a further feature, essential to the invention, of the product is that the total weight ratio G2/G4 has a value of 0.5 to 2.0. The total amount by weight G2 is understood to mean the total weight of all fatty components included in formulation (a). The calculation basis in this case is the total weight of preparation (a).

The total amount by weight G4 is understood to mean the total weight of all fatty components included in formulation (b). The calculation basis in this case is the total weight of preparation (b).

If the weight ratio of the fatty components in formulations (a) and (b) is within the value range of 0.5 to 2.0, both formulations can be dispensed reproducibly and constantly and via outlet opening of dispenser (C); i.e., it is possible to remove defined and constant amounts of formulations (a) and (b) over the entire application period. An application mixture, prepared from (a) and (b) and having the same composition in each removal step, can be removed in this way via the dispenser, regardless of whether the dispenser is still completely filled or has already been partially emptied. In this regard, the dispensing is the more precise, the closer the weight ratio G2/G4 is to the value of 1.

A very especially preferred product for the oxidative coloring and/or lightening of keratinic fibers is characterized further in that the weight ratio G2/G4 has a value of 0.6 to 1.8, preferably of 0.7 to 1.6, more preferably of 0.8 to 1.4, and particularly preferably of 0.85 to 1.2.

A very especially preferred product for the oxidative coloring and/or lightening of keratinic fibers is characterized further in that the weight ratio G2/G4 has a value of 0.6 to 1.8, preferably of 0.7 to 1.6, more preferably of 0.8 to 1.5, and particularly preferably of 0.85 to 1.3.

Preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.6 to 1.8 and
the weight ratio G2/G4 has a value of 0.6 to 1.8.

Preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.65 to 1.6 and
the weight ratio G2/G4 has a value of 0.7 to 1.6.

Preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.7 to 1.4 and
the weight ratio G2/G4 has a value of 0.8 to 1.4.

Preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.7 to 1.4 and
the weight ratio G2/G4 has a value of 0.85 to 1.2.

Within the scope of work leading to this invention, it has emerged that the total amounts by weight of the thickening polymers (G1 and G3) and the total amounts by weight of the fatty components (G2 and G4) also exert a mutual effect on one another.

The best results in regard to solving the problem addressed by the invention were obtained, when the thickening polymers and the fatty components were used in preparations (a) and (b) in such total amounts that the sum of the total weight of the thickening polymers and the fatty components in both formulations was also the same in each case, i.e., when the weight ratio (G1+G2)/(G3+G4) was also in a value range of 0.3 to 3.0. The closer this weight ratio as well approached a value of 1, the more homogeneous were the application mixtures obtained during use of the product of the invention and the more uniform as well was the color result obtained in the dyeing.

A furthermore very especially preferred product for the oxidative coloring and/or lightening of keratinic fibers is characterized in that the weight ratio (G1+G2)/(G3+G4) has a value of 0.3 to 3.0, preferably of 0.4 to 2.5, more preferably of 0.5 to 2.0, and particularly preferably of 0.7 to 1.4.

Preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.65 to 1.6,
the weight ratio G2/G4 has a value of 0.7 to 1.6, and
the weight ratio (G1+G2)/(G3+G4) has a value of 0.7 to 1.4.

Preferably preparations (a) and (b) include thickening polymers from the group of polysaccharides, acrylic acid polymers, acrylic acid copolymers, methacrylic acid polymers, and/or methacrylic acid copolymers. If preparations (a) and (b) include one or more thickening polymers from said group, the viscosity of both preparations can be matched especially well to one another. In particular, the viscosity of both preparations remains stable for rather long storage periods as well.

In a further especially preferred embodiment, a product of the invention is therefore characterized in that
preparation (a) includes as thickening polymer(s) one or more polymers from the group including polysaccharides, acrylic acid polymers, acrylic acid copolymers, methacrylic acid polymers, and/or methacrylic acid copolymers and
preparation (b) includes as thickening polymer(s) one or more polymers from the group including polysaccharides, acrylic acid polymers, acrylic acid copolymers, methacrylic acid polymers, and/or methacrylic acid copolymers.

It has emerged surprisingly that the rheological properties of preparations (a) and (b) can be matched especially well to one another by the use of special polysaccharides. In other words, preparations (a) and (b), both of which in each case include at least one special polysaccharide, whereby the total weight ratio of the thickening polymers G1/G3 has a value of 0.5 to 2.0, can be removed in an especially constant composition from the dispenser over the entire application period. Therefore, both preparations (a) and (b), although they can otherwise differ greatly from one another with respect to their pH and their ingredients, if special polysaccharides are added, have virtually identical rheological properties, which depend in particular also on the pressure within the dispenser and on the degree of dispenser filling.

The term polysaccharides is the collective term for macromolecular carbohydrates whose molecules consist of a large number of glycosidically linked monosaccharide molecules (with a molar mass of at least 5000 g/mol). These carbohydrates can also be chemically modified.

Xanthan gums, alginates, carboxyalkylcelluloses, and hyaluronic acids have a very particular suitability within the group of the polysaccharides.

Xanthan gum is a polysaccharide, which is made up of, inter alia, the structural components: D-glucose, D-mannose, D-glucuronic acid, acetate, and pyruvate, and is also known under the INCI name Xanthan Gum. Xanthan gum carries carboxy groups and is anionic or anionizable. The physiologically acceptable salts of xanthan are also novel.

Salts of alginic acid are called alginates (INCI name Algin). Alginates are acidic, carboxy group-including polysaccharides, consisting of D-mannuronic acid and D-guluronic acid in different ratios, which are linked by 1-4-glycosidic bonds. The alkali and alkaline earth salts of alginic acids in particular are novel. The use of alginic acid, sodium alginate, potassium alginate, ammonium alginate, and/or calcium alginate has proven especially advantageous in the preparations of the invention.

Carboxyalkylcelluloses are cellulose ethers in which the hydrogen atoms of the hydroxy groups of cellulose are partially or completely substituted by carboxyalkyl groups. A preferred carboxyalkylcellulose is carboxymethylcellulose, which preferably can be used as an anionic polymer in the form of its sodium salt (sodium carboxymethylcellulose).

The basic structural unit of hyaluronic acid (INCI names: Hyaluronic acid, Sodium Hyaluronate) is an amino disaccharide which is made up of D-glucuronic acid and N-acetylglucosamine in a 1-3-glycosidic bond and is connected to the next unit with a β-1-4-glycosidic bond. The sodium and potassium salts of hyaluronic acid have also proven especially suitable within the scope of work leading to this invention.

An especially preferred product for the oxidative coloring and/or lightening of keratinic fibers, therefore, is characterized furthermore in that
preparation (a) includes as thickening polymer(s) one or more polysaccharides from the group including carboxymethylcelluloses, alginic acids, xanthan gum, and/or the physiologically acceptable salts thereof, especially preferably xanthan gum and/or the physiologically acceptable salts thereof, and
preparation (b) includes as thickening polymer(s) one or more polysaccharides from the group including carboxymethylcelluloses, alginic acids, xanthan gum, and/or the physiologically acceptable salts thereof, especially preferably xanthan gum and/or the physiologically acceptable salts thereof.

It can be of particular advantage with respect to the matching of the flow behavior of preparations (a) and (b), if preparations (a) and (b) include the same thickening polymers. In another especially preferred embodiment, a product for the oxidative coloring and/or lightening of keratinic fibers is characterized in that preparation (a) and preparation (b) include the same thickening polymers.

Xanthan gum and/or the physiologically acceptable salts are particularly preferred in this regard, whereby it can also be preferable, if preparations (a) and (b) include no other thickening polymers apart from xanthan gum or the physiologically acceptable salts thereof.

Likewise preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.5 to 2.0 and
the weight ratio G2/G4 has a value of 0.5 to 2.0,
preparation (a) includes as the thickening polymer xanthan gum and/or the physiologically acceptable salt thereof and
preparation (b) includes as the thickening polymer xanthan gum and/or the physiologically acceptable salt thereof.

Likewise preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.5 to 2.0 and
the weight ratio G2/G4 has a value of 0.5 to 2.0,
preparation (a) includes as the thickening polymer xanthan gum and/or the physiologically acceptable salt thereof and
preparation (b) includes as the thickening polymer xanthan gum and/or the physiologically acceptable salt thereof,
preparations (a) and (b) include no other thickening polymers apart from xanthan gum and/or the physiologically acceptable salts thereof.

Preparations (a) and (b) can include as thickening polymer also at least one acrylic acid polymer and/or at least one methacrylic acid polymer. An acrylic acid polymer is understood to mean a homopolymer of acrylic acid or a homopolymer of an acrylic acid derivative. A methacrylic acid polymer is understood to mean a homopolymer of methacrylic acid or a homopolymer of a methacrylic acid derivative.

Preferred acrylic acid and methacrylic acid polymers include as repeat units structures of the formula (P-I)

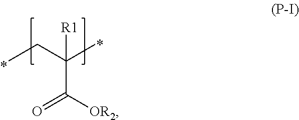

where
R1 stands for a hydrogen atom or a methyl group and
R2 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n stands for a number from 1 to 30.

In the case of the preferred acrylic acid polymers, R1 stands for a hydrogen atom, and in the case of the preferred methacrylic acid polymers, R1 stands for a methyl group.

A preferred homopolymer of acrylic acid is a polyacrylic acid, as can be obtained, for example, from the company 3V Sigma under the trade names Synthalen K or Synthalen M or from the company Lubrizol under the trade names Carbopol (for example, Carbopol 980, 981, 954, 2984, and 5984), in each case with the INCI name Carbomer. The compound sold by BASF under the trade name Cosmedia SP (INCI Name: SODIUM POLYACRYLATE) can also be cited as a preferred acrylic acid homopolymer in this regard.

Likewise preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.5 to 2.0 and
the weight ratio G2/G4 has a value of 0.5 to 2.0, and
preparation (a) includes as thickening polymer(s) at least one acrylic acid and/or methacrylic acid polymer with repeat units of the formula (P-I)

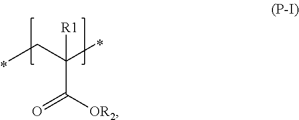

where
R1 stands for a hydrogen atom or a methyl group and
R2 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n for a number of 1 to 30 stands and
   preparation (b) includes as thickening polymer(s) at least one acrylic acid and/or methacrylic acid polymer with repeat units of the formula (P-I*)

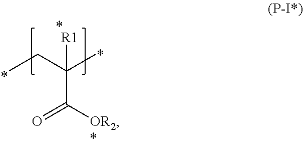

where
R1* stands for a hydrogen atom or a methyl group and
R2* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n stands for a number from 1 to 30.

Furthermore, preparations (a) and (b) can also include as thickening polymer at least one acrylic acid copolymer and/or a methacrylic acid copolymer.

Acrylic acid copolymers are understood to mean copolymers that are prepared by polymerization of acrylic acid or acrylic acid derivatives with one or more monomers different from acrylic acid or acrylic acid derivatives. Methacrylic acid copolymers are understood to mean copolymers that are prepared by polymerization of methacrylic acid or methacrylic acid derivatives with one or more monomers different from methacrylic acid or methacrylic acid derivatives.

Preferred acrylic acid and methacrylic acid copolymers include as repeat units structures of the formulas (P-II) and (P-III)

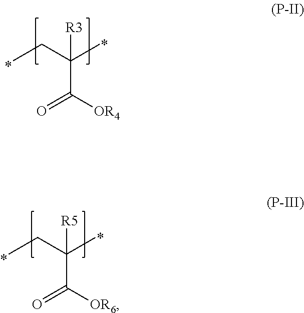

where
R3, R5 independently of one another stand for a hydrogen atom or a methyl group,
R4 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30.

R3 and R5 are preferably different from one another and/or R4 and R6 are different from one another.

Likewise preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.5 to 2.0 and
the weight ratio G2/G4 has a value of 0.5 to 2.0,
preparation (a) includes as thickening polymer(s) at least one acrylic acid copolymer and/or a methacrylic acid copolymer with repeat units of the formulas (P-II) and (P-III)

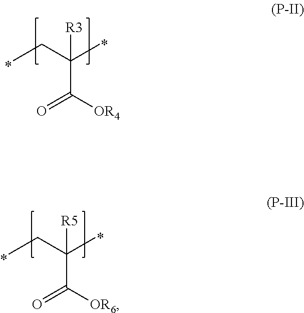

where
R3, R5 independently of one another stand for a hydrogen atom or a methyl group,
R4 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30, and
   preparation (b) includes as thickening polymer(s) at least one acrylic acid copolymer and/or a methacrylic acid copolymer with repeat units of the formulas (P-II*) and (P-III*)

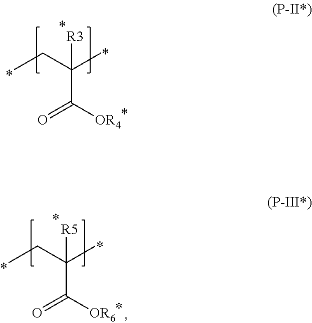

where
R3*, R5* independently of one another stand for a hydrogen atom or a methyl group,
R4* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30.

Preferred acrylic acid and methacrylic acid copolymers are the sodium acrylate/sodium acryloyldimethyl taurate copolymers sold under the name Simulgel® EG as a compound with isohexadecane and polysorbate 80 (INCI name: SODIUM ACRYLATE/SODIUM ACRYLOYLDIMETHYL TAURATE COPOLYMER, ISOHEXADECANE, POLYSORBATE 80).

A polymer further suitable in this regard is the polymer known under the INCI name Acrylates/C10-30 Alkyl Acrylate Crosspolymer, which can be obtained under the trade name Carbopol 1382 from the company Noveon. A further suitable polymer is the polymer known under the INCI name Acrylates/Steareth-20 Methacrylate Crosspolymer known polymer, which for example, with the trade name Aculyn® 88 from the company Rohm & Haas in the form of a 28 to 30% by weight dispersion in water. Further, polymers known according to the INCI nomenclature as Acrylates/Palmeth-25 Acrylate Copolymer or Acrylates/Palmeth-20 Acrylate Copolymer can be used. Such polymers can be obtained, for example, under the trade name Synthalen® W 2000 as a 30 to 32% by weight emulsion in water from the company 3 V Sigma.

It can also be preferred to use as the acrylic acid and/or methacrylic acid copolymer a copolymer of at least one anionic acrylic acid or methacrylic acid monomer and at least one non-ionogenic monomer. Preferred non-ionogenic monomers in this regard are acrylamide, methacrylamide, acrylic acid esters, methacrylic acid esters, vinylpyrrolidone, vinyl ethers, and vinyl esters.

Other preferred anionic copolymers are, for example, copolymers of acrylic acid and/or methacrylic acid, and/or the C₁-C₆ alkyl esters thereof, as they are sold under the INCI declaration Acrylates Copolymers. A preferred commercial product is, for example, Aculyn® 33 from the company Rohm & Haas. Preferred further, however, are also copolymers of acrylic acid and/or methacrylic acid, C₁-C₆ alkyl esters of acrylic acid and/or methacrylic acid, as well as esters of an ethylenically unsaturated acid and an alkoxylated fatty alcohol. Suitable ethylenically unsaturated acids are in particular acrylic acid, methacrylic acid, and itaconic acid; suitable alkoxylated fatty alcohols are in particular Steareth-20 or Ceteth-20. Copolymers of this type are sold by the company Rohm & Haas under the trade name Aculyn® 22 (INCI name: Acrylates/Steareth-20 Methacrylate Copolymer).

Other preferred acrylic acid or methacrylic acid copolymers are acrylic acid-acrylamide copolymers.

Preparations (a) and (b) can also include the following zwitterionic acrylic acid or methacrylic acid polymers:
copolymers of dimethyldiallylammonium salts and acrylic acid, e.g., Polyquaternium-22,
copolymers of dimethyldiallylammonium salts and methacrylic acid,
   copolymers of N,N,N-trimethyl-3-[(2-methyl-1-oxo-2-propen-1-yl)amino]-1-propanaminium salts and acrylic acid,
   copolymers of N,N,N-trimethyl-3-[(2-methyl-1-oxo-2-propen-1-yl)amino]-1-propanaminium salts and methacrylic acid,
   copolymers of N,N,N-trimethyl-2-[(2-methyl-1-oxo-2-propen-1-yl)amino]-1-ethanaminium salts and acrylic acid,
   copolymers of N,N,N-trimethyl-2-[(2-methyl-1-oxo-2-propen-1-yl)amino]-1-ethanaminium salts and methacrylic acid,
   copolymers of N,N,N-trimethyl-3-[(2-methyl-1-oxo-2-propen-1-yl)amino]-1-propanaminium salts, acrylic acid, and acrylamide, e.g., Polyquaternium-53,
   copolymers of N,N,N-trimethyl-3-[(2-methyl-1-oxo-2-propen-1-yl)amino]-1-propanaminium salts, methacrylic acid, and acrylamide,
copolymers of 1-ethenyl-3-methyl-1H-imidazolium salts, 1-ethenyl-1H-imidazole, 1-ethenyl-2-pyrrolidinone, and methacrylic acid, e.g., Polyquaternium-86,
copolymers of 1-ethenyl-3-methyl-1H-imidazolium salts, 1-ethenyl-1H-imidazole, 1-ethenyl-2-pyrrolidinone, and acrylic acid.

Preparations (a) and (b) of the invention can also include mixtures of the aforementioned preferred thickening polymers, whereby the requirement again applies that the ratio of the total amounts by weight of the thickening polymers (G1) and (G3) in preparations (a) and (b) has a value of 0.5 to 2.0.

Over the course of the work leading to this invention, it emerged that it is especially advantageous, if the thickening polymers in preparations (a) and (b) are each used in specific amount ranges.

In this case, the complete emptying of the dispenser via outlet opening (C) could be assured particularly when the total amounts by weight (G1) and (G3) of the thickening polymers in preparations (a) and (b) in each case were in an amount range between 0.7 and 2.5% by weight. If the total amounts by weight (G1) and (G3) were selected too high (e.g., above 9.5% by weight), clumps occurred within the dispenser or during removal. If the total amounts by weight (G1) and (G3), in contrast, were selected as too low, the matching of the rheological properties of the two preparations (a) and (b) was only insufficiently possible, so that the composition of the application mixture (of (a) and (b)) changed over the course of the application process (i.e., with increasing emptying of the dispenser).

In another especially preferred embodiment, a product for the oxidative coloring and/or lightening of keratinic fibers is characterized in that
preparation (a) includes the thickening polymer(s) in a total amount by weight (G1) of 0.5 to 9.5% by weight, preferably of 0.6 to 7% by weight, more preferably of 0.7 to 4.5% by weight, and particularly preferably of 0.7 to 2.5% by weight, based on the total weight of preparation (a), and
preparation (b) includes the thickening polymer(s) in a total amount by weight (G3) of 0.5 to 9.5% by weight, preferably of 0.6 to 7% by weight, more preferably of 0.7 to 4.5% by weight, and particularly preferably of 0.7 to 2.5% by weight, based on the total weight of preparation (b).

A further feature essential to the invention is the matching of preparations (a) and (b) in regard to their respective total content of fatty components.

Preferred fatty components can be understood to mean those from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons. Only nonionic substances are regarded explicitly as fatty components within the meaning of the present invention. Charged compounds such as, for example, fatty acids and salts thereof are not understood to be a fatty component.

C₁₂-C₃₀ fatty alcohols can be saturated, mono- or polyunsaturated, linear or branched fatty alcohols having 12 to 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

Preferred representatives of branched fatty alcohols are 2-octyldodecanol, 2-hexyldodecanol, and/or 2-butyldodecanol.

A C₁₂-C₃₀ fatty acid triglyceride within the meaning of the present invention is understood to mean the triesters of the trihydric alcohol glycerol with three equivalents of fatty acid. In this regard, both structurally similar and also different fatty acids can be involved in ester formations within a triglyceride molecule.

Fatty acids according to the invention are understood to mean saturated or unsaturated, unbranched or branched, unsubstituted or substituted C₁₂-C₃₀ carboxylic acids. Unsaturated fatty acids can be mono- or polyunsaturated. In the case of an unsaturated fatty acid, the C—C double bond(s) thereof can have the cis or trans configuration.

The fatty acid triglycerides are notable for a particular suitability in which at least one of the ester groups originating from glycerol is formed with a fatty acid, selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,13Z)-eicosa-5,8,11,14-tetraenoic acid], and/or nervonic acid [(15Z)-tetracos-15-enoic acid].

The fatty acid triglycerides can also be of natural origin. The fatty acid triglycerides, occurring in soybean oil, peanut oil, olive oil, sunflower oil, macadamia nut oil, Moringa oil, apricot kernel oil, Marula oil, and/or optionally hydrogenated castor oil, or mixtures thereof are especially suitable for use in the product of the invention.

A C₁₂-C₃₀ fatty acid monoglyceride is understood to mean the monoester of the trihydric alcohol glycerol with a fatty acid equivalent. In this case, either the middle hydroxy group of glycerol or the terminal hydroxy group of glycerol can be esterified with the fatty acid.

The C₁₂-C₃₀ fatty acid monoglycerides are notable for a particular suitability, in which a hydroxy group of glycerol is esterified with a fatty acid, whereby the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

A C₁₂-C₃₀ fatty acid diglyceride is understood to mean the diester of the trihydric alcohol glycerol with two fatty acid equivalents. In this case, either the middle and a terminal hydroxy group of glycerol can be esterified with two fatty acid equivalents, or however both terminal hydroxy groups of glycerol are esterified with one fatty acid in each case. Glycerol can be esterified hereby both with two structurally similar and with two different fatty acids.

The fatty acid diglycerides are notable for a particular suitability in which at least one of the ester groups originating from glycerol is formed with a fatty acid, selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,13Z)-eicosa-5,8,11,14-tetraenoic acid], and/or nervonic acid [(15Z)-tetracos-15-enoic acid].

Hydrocarbons are compounds consisting exclusively of carbon and hydrogen atoms and having 8 to 80 C atoms. Preferred in this regard are particularly aliphatic hydrocarbons such as, for example, mineral oils, liquid paraffin oils (e.g., liquid paraffin or light liquid paraffin), isoparaffin oils, semisolid paraffin oils, paraffin waxes, hard paraffin (solid paraffin), Vaseline, and polydecene.

Liquid paraffin oils (liquid paraffin and light liquid paraffin) have proven to be especially suitable in this regard. The hydrocarbon is very especially preferably liquid paraffin, also called white oil. Liquid paraffin is a mixture of purified, saturated, aliphatic hydrocarbons, which consists for the most part of hydrocarbon chains with a C-chain distribution of 25 to 35 C atoms.

The more greatly preparations (a) and (b) differ with respect to the ingredients included additionally in both preparations, the more difficult it is typically to match the flow behavior of the two preparations. If preparations (a) and (b) differ greatly in regard to the ingredients they include in addition, the flow properties of both preparations nonetheless can be matched to one another very well and reproducibly, if the preparations include one or more compounds from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons. Preferred in this regard are C₁₂-C₃₀ fatty alcohols and/or hydrocarbons, C₁₂-C₃₀ fatty alcohols being very especially preferred.

In another especially preferred embodiment, a product for the oxidative coloring and/or lightening of keratinic fibers is characterized in that
preparation (a) includes as the fatty component(s) one or more compounds from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons, preferably C₁₂-C₃₀ fatty alcohols and/or hydrocarbons, particularly preferably C₁₂-C₃₀ fatty alcohols, and
preparation (b) includes as the fatty component(s) one or more compounds from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons, preferably C₁₂-C₃₀ fatty alcohols and/or hydrocarbons, particularly preferably C₁₂-C₃₀ fatty alcohols.

The rheological properties of formulations (a) and (b) can also be matched especially well and reproducibly to one another, if the fatty components in both formulations (a) and (b) are used in precisely defined amount ranges.

In another especially preferred embodiment, a product for the oxidative coloring and/or lightening of keratinic fibers is characterized in that
preparation (a) includes the fatty component(s) in a total amount by weight (G2) of 0.25 to 9.5% by weight, preferably of 0.4 to 8.5% by weight, more preferably of 0.6 to 7.5% by weight, and particularly preferably of 1.0 to 6.0% by weight, based on the total weight of preparation (a), and
preparation (b) includes the fatty component(s) in a total amount by weight (G4) of 0.25 to 9.5% by weight, preferably of 0.4 to 8.5% by weight, more preferably of 0.6 to 7.5% by weight, and particularly preferably of 1.0 to 6.0% by weight, based on the total weight of preparation (b).

In another especially preferred embodiment, a product for the oxidative coloring and/or lightening of keratinic fibers is characterized in that
preparation (a) includes the fatty component(s) in a total amount by weight (G2) of 0.25 to 9.5% by weight, preferably of 0.4 to 8.5% by weight, more preferably of 0.6 to 8.0% by weight, and particularly preferably of 1.0 to 7.5% by weight, based on the total weight of preparation (a), and
preparation (b) includes the fatty component(s) in a total amount by weight (G4) of 0.25 to 9.5% by weight, preferably of 0.4 to 8.5% by weight, more preferably of 0.6 to 8.0% by weight, and particularly preferably of 1.0 to 7.5% by weight, based on the total weight of preparation (b).

The use of thickening polymers and fatty components in amounts by weight matched to one another in preparations (a) and (b) assures the removal of constant quantity ratios of both formulations via outlet opening (C), because in this way the rheological properties of both formulations can be matched reliably to one another. The use of the aforementioned special representatives from the group of the polymeric thickeners and the fatty components assures hereby primarily a similar flow behavior, also little affected by differences in dispenser geometry, in preparations (a) and (b), which otherwise have a very different composition. In this case, the maintenance of special viscosity ranges has proven to be very especially advantageous.

Very especially preferred therefore is a product for the oxidative color changing of keratinic fibers, which is characterized in that
preparation (a) has a viscosity of 1000 to 100,000 mPas, preferably of 2000 to 80,000 mPas, more preferably of 4000 to 60,000 mPas, even more preferably of 7000 to 40,000 mPas, and particularly preferably of 10,000 to 30,000 mPas (22° C./Brookfield viscometer/spindle 5/4 rpm),
preparation (b) has a viscosity of 1000 to 100,000 mPas, preferably of 2000 to 80,000 mPas, more preferably of 4000 to 60,000 mPas, even more preferably of 7000 to 40,000 mPas, and particularly preferably of 10,000 to 30,000 mPas (22° C./Brookfield viscometer/spindle 5/4 rpm), and
the ratio of the viscosities V1/V2 has a value of 0.3 to 3.0, preferably of 0.5 to 2.0, more preferably of 0.6 to 1.8, and particularly preferably of 0.8 to 1.3.

The indicated viscosities are viscosities that were measured at 22° C. (22° C./Brookfield viscometer/spindle 5/4 rpm).

Very especially preferred furthermore, therefore, is a product for the oxidative color changing of keratinic fibers, which is characterized in that
preparation (a) has a viscosity of 1000 to 100,000 mPas, preferably of 2000 to 80,000 mPas, more preferably of 4000 to 60,000 mPas, even more preferably of 7000 to 50,000 mPas, and particularly preferably of 10,000 to 45,000 mPas (22° C./Brookfield viscometer/spindle 5/4 rpm),
preparation (b) has a viscosity of 1000 to 100,000 mPas, preferably of 2000 to 80,000 mPas, more preferably of 4000 to 60,000 mPas, even more preferably of 7000 to 50,000 mPas, and particularly preferably of 10,000 to 45,000 mPas (22° C./Brookfield viscometer/spindle 5/4 rpm), and
the ratio of the viscosities V1/V2 has a value of 0.3 to 3.0, preferably of 0.5 to 2.0, more preferably of 0.6 to 1.8, and particularly preferably of 0.8 to 1.3.

The indicated viscosities are viscosities that were measured at 22° C. (22° C./Brookfield viscometer/spindle 5/4 rpm).

Preparations (a) and/or (b) can still include other additional ingredients.

If not just lightening but oxidative coloring as well are desired, preparation (a) can include in addition also one or more oxidation dye precursors, preferably at least one oxidation dye precursor of the developer type and at least one oxidation dye precursors of the coupler type. Especially suitable oxidation dye precursors of the developer type are thereby selected from at least one compound from the group, formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one, and the physiologically acceptable salts thereof.

Especially suitable oxidation dye precursors of the coupler type in this case are selected from the group, formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

In addition, preparation (a) can also include at least one direct dye from the group of anionic, nonionic, and/or cationic dyes.

Especially preferably, this concerns one or more nonionic direct dyes from the group including HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

In another especially preferred embodiment, preparation (a) is characterized in that it includes in addition one or more nonionic direct dyes from the group including HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

In addition, anionic direct dyes may also be present, which are known under the international names or trade names: Acid Yellow 1, Yellow 10, Acid Yellow 23, Acid Yellow 36, Acid Orange 7, Acid Red 33, Acid Red 52, Pigment Red 57:1, Acid blue 7, Acid Green 50, Acid Violet 43, Acid Black 1, Acid Black 52, bromophenol blue, and tetrabromophenol blue.

Suitable cationic direct dyes are cationic triphenylmethane dyes, such as, for example, Basic Blue 7, Basic Blue 26, Basic Violet 2, and Basic Violet 14, aromatic systems, substituted with a quaternary nitrogen group, such as, for example, Basic Yellow 57, Basic Red 76, Basic Blue 99, Basic Brown 16, and Basic Brown 17, cationic anthraquinone dyes, such as HC Blue 16 (Bluequat B), and direct dyes, including a heterocycle that has at least one quaternary nitrogen atom, particularly Basic Yellow 87, Basic Orange 31, and Basic Red 51. The cationic direct dyes, marketed under the trademark Arianor, are also suitable cationic direct dyes according to the invention.

The oxidation dye precursors, i.e., developer components and coupler components, and the optional additional direct dyes can be used, for example, in an amount of 0.0001 to 5.0% by weight, preferably 0.001 to 3.5% by weight, in each case based on the total weight of preparation (a).

A surface-active substance is preferably added in addition to preparations (a) and (b), whereby such surface-active substances are called surfactants or emulsifiers depending on the field of application: they are preferably selected from anionic, cationic, zwitterionic, amphoteric, and nonionic surfactants and emulsifiers.

Preparations (a) and (b) preferred according to the invention are characterized in that they include in addition at least one anionic surfactant. Preferred anionic surfactants are fatty acids, alkyl sulfates, alkyl ether sulfates, and ether carboxylic acids having 10 to 20 C atoms in the alkyl group and up to 16 glycol ether groups in the molecule. The anionic surfactants are used in amounts of 0.1 to 45% by weight, preferably 1 to 30% by weight, and very especially preferably of 1 to 15% by weight, based on the total amount of the ready-to-use agent.

Preparations (a) and (b) preferred according to the invention are characterized in that preparations (a) and/or (b) include in addition at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethylimidazolines. A preferred zwitterionic surfactant is known by the INCI name Cocamidopropyl Betaine.

Preparations (a) and (b) preferred according to the invention are characterized in that preparations (a) and/or (b) include in addition at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids. Especially preferred amphoteric surfactants are N-cocoalkyl aminopropionate, cocoacylaminoethyl aminopropionate, and C₁₂-C₁₈ acylsarcosine.

The nonionic, zwitterionic, or amphoteric surfactants can be used in proportions of 0.1 to 40% by weight, preferably 1 to 30% by weight, and very particularly preferably of 1 to 15% by weight, based in each case on the total amount of preparations (a) and (b).

It has proven advantageous, furthermore, for the agents to include other non-ionogenic surface-active substances. Preferred nonionic surfactants are, for example, alkylene oxide adducts to fatty alcohols and fatty acids with in each case 2 to 30 mol of ethylene oxide per mole of fatty alcohol or fatty acid. Preparations with excellent properties are likewise obtained if they include fatty acid esters of ethoxylated glycerol as the nonionic surfactants. Nonionic surfactants have a solubility in water at room temperature (22° C.) and atmospheric pressure (760 mm Hg) of more than 0.1% by weight, preferably of more than 1.0% by weight.

Preparation (a) is preferably a formulation that is made alkaline and optionally includes the oxidation dye precursors, if a color change is also desired in addition to the lightening. Preparation (b) is a formulation with oxidizing agents that is made acidic for reasons of stability.

In another especially preferred embodiment, a product of the invention is therefore characterized in that
preparation (a) includes at least one alkalinizing agent, preferably ammonia and/or monoethanolamine, and
preparation (b) includes at least one oxidizing agent, preferably hydrogen peroxide.

Preparation (a) preferably includes at least one alkalinizing agent, preferably ammonia and/or monoethanolamine. Alkalinizing agents that can be used according to the invention can be selected from the group that is formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. Suitable inorganic alkalinizing agents are sodium hydroxide, potassium hydroxide, sodium silicate, and sodium metasilicate. Organic alkalinizing agents that can be used according to the invention can be selected from monoethanolamine, 2-amino-2-methylpropanol, and triethanolamine. The basic amino acids that can be used as alkalinizing agents according to the invention are preferably selected from the group formed by arginine, lysine, ornithine, and histidine, especially preferably arginine.

Ammonia (NH₃) is customarily employed in the form of its aqueous solution. Aqueous ammonia solutions include ammonia (NH₃) often in concentrations between 10% by weight and 32% by weight. Preferred in this case is the use of an aqueous ammonia solution, which includes 25% by weight of ammonia (NH₃).

Preferably, preparation (a) includes ammonia and/or monoethanolamine in amounts of 0.01 to 10% by weight, preferably of 0.1 to 7.5% by weight, more preferably of 0.2 to 5.5% by weight, and particularly preferably of 0.4 to 4.5% by weight, in each case based on the total weight of preparation (a).

Coloring processes on keratin fibers typically take place in an alkaline environment. To treat keratin fibers and the skin as well as gently as possible, setting a too high pH is not desirable, however. It is preferred, therefore, if the pH of the ready-to-use agent (i.e., the mixture of preparations (a) and (b)) is between 7 and 11 and particularly between 8 and 10.5. pH values within the meaning of the present invention are pH values measured at a temperature of 22° C.

In this case, preparation (a) is preferably adjusted to an alkaline pH, and preparation (b) advantageously has an acidic pH.

In another especially preferred embodiment, a product of the invention is characterized in that
preparation (a) has a pH of 8 to 12.5, preferably of 8.5 to 12, more preferably of 9 to 11.5, and particularly preferably of 9.5 to 11 and
preparation (b) has a pH of 1 to 7, preferably of 1.5 to 6, more preferably of 2.5 to 5.5, and particularly preferably of 3.0 to 5.0.

Hydrogen peroxide is preferably used as an aqueous solution in preparation (b). The concentration of a hydrogen peroxide solution in the agent of the invention is determined, on the one hand, by legal requirements and, on the other, by the desired effect; preferably, 6 to 12% by weight solutions in water are used. Preparations (b) preferred according to the invention are characterized in that, based on the total weight of preparation (b), they include 0.5 to 20% by weight, preferably 1 to 12.5% by weight of hydrogen peroxides, based in each case on the total weight of the agent.

Preparations (a) and (b) furthermore can include additional active substances, auxiliary substances, and additives in order to improve the coloring or lightening performance and to set other desired properties of the agents.

It has proven advantageous, if the dyes (i.e., preparations (a) and particularly (b)), particularly if they include additional hydrogen peroxide, include at least one stabilizer or complexing agent. Especially preferred stabilizers are phenacetin, alkali benzoates (sodium benzoate), salicylic acid, and dipicolinic acid. Furthermore, all complexing agents in the state of the art can be used. Complexing agents preferred according to the invention are nitrogen-including polycarboxylic acids, particularly EDTA and EDDS, and phosphonates, particularly 1-hydroxyethane-1,1-diphosphonate (HEDP), and/or ethylenediamine tetramethylene phosphonate (EDTMP), and/or diethylenetriamine pentamethylene phosphonate (DTPMP), or sodium salts thereof.

Further, the preparations of the invention can include other active substances, auxiliary substances, and additives, such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxy, alkoxy, and/or hydroxyl groups (dimethicone copolyols), linear polysiloxanes(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂, and air.

The selection of these additional substances is made by the skilled artisan according to the desired properties of preparations (a) and (b). In regard to other facultative components and the employed amounts of said components, reference is made expressly to relevant handbooks known to the skilled artisan. The additional active and auxiliary substances are used in the agents of the invention preferably in each case in amounts of 0.0001 to 25% by weight, particularly of 0.0005 to 15% by weight, based on the total weight of the application mixture.

The dispenser within the meaning of the present invention is understood to mean a container including at least two chambers ((A) and (B)), which can be fabricated, for example, from plastic, metal, and/or plastic-metal composites. The dispenser can have, inter alia, the shape of a box, a bottle, a container, a pressurized container, or a tube. Suitable for the product of the invention are all dispenser designs that assure that the product compositions (a) and (b) present in both chambers of the dispenser are kept reliably isolated from another until removal.

Preferably, the individual product compositions are combined to form a total formulation during the product applications, in order to exploit in this way the full effectiveness of the total formulation and to make product use easier in general. For this purpose, the dispenser expediently includes, in addition to chambers (A), (B), a dispensing head within which the product compositions (a), (b) are conveyed from the chambers to outlet opening (C). In this regard, a suitable mixing device is formed in the dispensing head; upstream from outlet opening (C), said device provides for the desired mixing of the product compositions (a), (b), before the mixed total formulation, i.e., (a)+(b), is dispensed via outlet opening (C). For example, a generic dispenser with a comparable mixing device is known from DE 3729491 A1, whereby the mixing device therein only has a very short mixing section, however. Such a mixing device according to the invention is structurally integrated directly into the dispensing head or alternatively, however, disposed as a separate structural element within the dispensing head. Static mixers or comparably acting mixing sections, for example, are to be understood to mean suitable mixing devices within the meaning of the invention; these are traversed by the free-flowing product compositions (a), (b) and sufficiently mixed during this through-flow. To this end, such a mixing section typically has suitable flow baffles or flow spoilers, which cause mixing of individual fluid components due to generated turbulences during the surround-flow. Critical for the quality of the mixing of individual fluid components, in this case the product compositions (a), (b), within the mixing device is, inter alia, the selective coordination of the length of the mixing section and the design of the flow baffles to the rheological properties of the product compositions (a), (b). Defined minimum lengths of the mixing section are usually necessary for an adequate mixing result of the mixing device of the invention. Therefore, the mixing section of the mixing device of the invention is preferably designed such that a defined minimum length of the mixing section is guaranteed, without abandoning an overall compact construction of the mixing device and thus of the dispensing head. Thus, adequate mixing results are guaranteed and simultaneously the desired compact outer dimensions of the mixing device and of the dispensing head are assured. To this end, the mixing section is formed, for example, spiral-shaped or comparably compact within the dispensing head.

Chamber (B) of the dispenser can be disposed beside, above, or below chamber (A). Furthermore, chamber (B) can lie within chamber (A) or chamber (A) can lie within chamber (B), whereby in each case the one chamber encloses the other chamber partially or completely. The capacity of chambers (A) and (B) can be 10 cm³ to 1000 cm³ in each case, and the capacity of chambers (A) and (B) can be the same or different. Preferably, the capacity of chambers (A) and (B) is the same. Preferably, chambers A and B are arranged next to one another.

The dispenser has an outlet opening (C), which is in fluid communication with both chambers (A) and (B) or can be brought into fluid communication with it via valves. When the application composition is removed, both formulations (a) and (b) are removed simultaneously via the common outlet opening and are preferably already mixed together upstream by means of the above-described mixing device. The usually reactive individual formulations (a) and (b) thereby come into contact for the first time during product use and hereby form the ready-to-use mixture.

Outlet opening (C) can be, for example, a pressurized container-dispensing device, a pump element, a valve, or a dispensing device for pasty materials. The removal of preparations (a) and (b) via common outlet opening (C) can occur by pressing the valve or by pumping the pump element.

The dispenser can also be a squeeze container; in this case, preparations (a) and (b) are removed preferably by pressing the squeeze container, whereby the pressure on the squeeze container results in the discharging of preparations (a) and (b) from common outlet opening (C).

Furthermore, all designs of an outlet opening (C) are also conceivable that enable formulations (a) and (b) to leave the dispenser together. An especially preferred product is characterized in that
the dispenser is a pressurized container,
outlet opening (C) is a pressurized container-dispensing device, which also has an appropriate mixing device, and that
the dispenser in addition includes at least one propellant from the group including propane, propene, n-butane, isobutane, isobutene, n-pentane, pentene, isopentane, isopentene, air, nitrogen, argon, N₂O, and/or CO₂.

In the aforementioned preferred embodiment, the product of the invention includes a pressurized container. Vessels made of metal (aluminum, tin plate, tin), protected or non-splintering plastic or of glass that is coated on the outside with plastic may be used as compressed-gas containers; pressure and breaking strength, corrosion resistance, easy fillability, as well as aesthetic aspects, handling, printability, etc., play a role in their selection. Special protective interior coatings can assure corrosion resistance against the preparation within the pressurized container if necessary.

Very especially preferably, the two chambers (A) and (B) separated from one another are two deformable pouches, preferably made of laminated aluminum, which are each in communication with outlet opening (C). Outlet opening (C) is a pressurized container-delivery device. Both pouches are in a box-shaped pressurized container, whereby the pressurized container together with the pressurized container-dispensing device close off the dispenser pressure-tight outwardly. The space between the outer wall of the pouch and the inner wall of the pressurized container is filled with at least one propellant. Suitable dispensers are known, for example, from US 2009/0108021 A1. A very especially preferred product for treating keratinic fibers is therefore characterized by such a dispenser with a suitable pressurized container, which has the above-described mixing device. A dispenser constructed in this way takes into account not only the chemical reactivity of the employed individual product compositions (a), (b), but moreover as a result of the mixing provides for a high effectiveness as well of the employed total formulation. Especially good effects according to the invention are achieved when the internal pressure of the pressurized container is at least 1.8 bar, particularly at least 2.5 bar.

The product includes furthermore a dispensing device (corresponding to outlet opening C), which can have a valve for discharging the application mixture. In a preferred embodiment of the invention, the valve has a valve disc with a coating or a polymeric plastic coating and just such a flexible element with a reset function, which after the operation ends resets the valve to the closed position (=neutral position of the valve). Corresponding cosmetic products in which the aerosol dispensing device includes a valve, which has a valve cone and/or a flexible element with reset function, which is/are covered with a coating or a polymeric plastic, are also preferred according to the invention.

In another preferred embodiment of the invention, the valve has a flexible element with a reset function and/or a valve disc made of at least one plastic, preferably a elastomeric plastic. Here as well, cosmetic products of the invention in which the valve has a flexible element with a reset function and/or a valve cone made of at least one plastic are preferred, whereby preferred plastics are elastomeric plastics. Especially preferred elastomeric plastics are selected from Buna, particularly Buna N, Buna 421, Buna 1602, and Buna KA 6712, neoprene, butyl, and chlorobutyl.

In another preferred embodiment of the invention, the flexible element with a reset function can be formed as a spiral spring or helical compression spring. In another preferred embodiment of the invention, the flexible element of the valve with the reset function can be formed integrally with the valve cone and have flexible legs. Such a spring can be made of metal or plastic.

All valves used according to the invention preferably have an internally coated valve disc, whereby the coating and valve material are compatible with one another. If aluminum valves are used according to the invention, thus the valve discs thereof can be coated on the inside, e.g., with a Micoflex coating. If tin plate valves are used according to the invention, thus the valve discs thereof can be coated on the inside, e.g., with PET (polyethylene terephthalate). The employed containers, which can be made, e.g., of tin plate or aluminum, aluminum containers being preferred according to the invention, must also be painted or coated on the interior because of the corrosivity of the water-in-oil emulsions, used as taught by the invention.

If the product of the invention is applied via a pressurized container, the dispensers include in addition at least one propellant from the group including propane, propene, n-butane, isobutane, isobutene, n-pentane, pentene, isopentane, isopentene, air, nitrogen, argon, N₂O, and/or CO₂. Within this group, the permanent gases: air, nitrogen, argon, N₂O, and/or CO₂ are preferred; nitrogen, argon, and/or CO₂ are very especially preferred.

Furthermore, it has proven to be preferable if the propellants are also kept at specific pressures in the dispenser. In a preferred embodiment, the dispensers of the invention include one or more additional propellants therefore with a pressure of 3 to 12 bar, preferably of 4 to 10 bar, and especially preferably of 5 to 8 bar, in each case based on the pressure of the propellants between the pouch-shaped chambers (A) and (B) and the pressurized container.

In another especially preferred embodiment, the dispenser is a dispenser for pasty materials, a multi-chamber tube, or a squeeze container.

In another very especially preferred embodiment, a product of the invention is characterized in that
the dispenser is a dispenser for pasty materials, a tube, or a squeeze tube and
outlet opening (C) is a pump element, a valve, or a dispensing device for pasty materials.

The product of the invention according to an alternative design form includes a multi-chamber tube, which has an inner and outer chamber, both of which end in a common outlet opening (C). The head area is designed such that the two preparations leave the tube together as soon as pressure is exerted on them. The design of said head region determines the pattern in which the preparations leave the tube. In principle, the present invention can comprise any distribution of chambers within the tube. In a first embodiment, for example, the two individual chambers can be arranged next to one another in an outer shell. In an embodiment especially preferred according to the invention, the multi-chamber tube, however, consists of an inner tube completely surrounded by an outer tube.

The multi-chamber tube is preferably fabricated of a material that is suitable for packaging oxidative color changing agents of this type. Laminated aluminum has proven especially suitable according to the invention both for the outer walls and for the inner walls. However, tubes made of plastic laminate (PE, PET, PP) or plastic coextrudates (PE, PET, PP) are also conceivable. Moreover, in one embodiment the material of the inner tube can be selected independently of the material of the outer tube. A tube in which the inner tube is fabricated of an aluminum laminate, which optionally is also protected with a coating, and the outer tube either of aluminum laminate or plastic laminate has proven very especially preferable according to the invention. Aluminum laminate according to the invention is understood to mean a plastic-coated aluminum layer.

It is especially advantageous if the shoulder region of the outer tube is reinforced with round blanks which have especially good barrier properties. In this regard, it is advantageous to incorporate aluminum into the material of the round blanks. To prevent the escape of the mixture during storage and to assure the consumer of the intactness of the tube, it is advantageous to seal the outlet opening with a tamper-evident closure made of aluminum or plastic which is removed by the user.

The selection of the volumes of the individual chambers (A) and (B) is determined by the desired ratio of the volumes of preparation (a) and preparation (b). The volumes of chambers (A) and (B) are preferably the same. The quantity ratio of preparation (a) to the amount of preparation (b) according to the invention is preferably in a range of 1:3 to 3:1; a range of 1:1.5 to 1.5:1 is preferred according to the invention, and a quantity ratio of 1:1 is especially preferred.

Likewise, very especially preferred is a product for the oxidative coloring and/or lightening of keratinic fibers, including

(1) a dispenser, which
has two chambers (A) and (B) separate from one another,
has an outlet opening (C), which is in communication with chamber (A) and with chamber (B),
   (2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
   (3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
   characterized in that
the weight ratio G1/G3 has a value of 0.5 to 2.0 and
the weight ratio G2/G4 has a value of 0.5 to 2.0,
the weight ratio of the total weight of preparation (a) to the total weight of preparation (b) is a value of 0.5 to 2.0, preferably of 0.6 to 1.8, more preferably of 0.7 to 1.4, and particularly preferably of 0.8 to 1.2.

The products of the invention can be used in methods for the oxidative changing of hair color. These methods distinguish themselves for the consumer by the particular ease of use, because there is no time-consuming and error-prone preparation of the application mixtures by the consumer. In addition, a particularly uniform color result is enabled with use of the products, because due to the special rheological matching of formulations (a) and (b) it is possible to remove the two formulations in precisely defined proportions, which are always the same relative to one another, during the entire application process.

The removal of the application mixture in this case usually occurs in batches, so that chambers (A) and (B) of the product are emptied stepwise in a defined ratio to one another. The consumer generally removes the amount of the application mixture, necessary for coloring or lightening one or more strands or sections of hair, from the dispenser, and then applies this amount to the hair. This process is repeated by the consumer until either the product has been totally emptied or the consumer has treated all of the hair.

In particular, a continuous removal of the product is also conceivable in the packaging of the product in the form of a pressurized container, however; i.e., in this case chambers (A) and (B) are emptied continuously by the operation of outlet opening (C), whereby here as well the composition of the application mixture (of preparations (a) and (b)) is the same and defined at each point in time of the removal.

A further subject matter of the present invention is a method for the coloring and/or lightening of keratinic fibers with the use of a product of the first subject matter of the invention, whereby
the dispenser is emptied continuously in one step or in batches in individual steps and
in each step defined, constant amounts of preparations (a) and (b) are removed each time from chambers (A) and (B) via common outlet opening (C) of the dispenser until both chambers (A) and (B) have been emptied completely.

A method is preferred in which chambers (A) and (B) are emptied in batches in a plurality of steps.

Especially preferred is a method for the coloring and/or lightening of keratinic fibers with the use of a product of the first subject matter of the invention, whereby
the dispenser is emptied in batches in a plurality of steps and
in each step defined, constant amounts of preparations (a) and (b) are removed each time from chambers (A) and (B) via common outlet opening (C) of the dispenser until both chambers (A) and (B) have been emptied completely.

It can be desirable in this regard that the same amounts of preparations (a) and (b) are removed in each step or each time. If one of chambers (A) or (B) has a larger volume or capacity, however, and/or if one of preparations (a) and (b) is present in a higher amount in the product, thus it can also be desirable to remove preparations (a) and (b) in each step in a constant quantity ratio of 3:1 to 1:3.

It is especially preferred if the amount of preparations (a) and (b), removed in steps, is the same, i.e., if the same amounts (a) and (b) are removed from the dispenser in each removal step.

A method for the coloring and/or lightening of keratinic fibers, therefore, is very especially preferred, which is characterized in that
in each step the same amounts of preparations (a) and (b) are removed each time from chambers (A) and (B) via common outlet opening (C) of the dispenser until both chambers (A) and (B) have been emptied completely.

The statements made regarding the product of the invention apply mutatis mutandis with respect to other preferred embodiments of the method of the invention.

### EXAMPLES

### Example 1

### 1. Preparation of Preparations (a) and (b)

The following preparations (a) and (b) were prepared (all quantities are given in % by weight of active substance):

| | | Prepa- | Prepa- |
|---|---|---|---|
| | | ration | ration |
| Ingredients | | (a) | (b) |
| Lanette D | Fatty component | 3.30 | 3.30 |
| (cetearyl alcohol) | | | |
| Lorol C12-C18 (techn.) | Fatty component | 1.20 | 1.20 |
| (C12-C18 fatty alcohols) | | | |
| Glyceryl oleate | Fatty component | 0.6 | 0.6 |
| Cocoglucosides | Nonionic emulsifier | 0.6 | 0.6 |
| Ceteareth-20 | Nonionic emulsifier | 0.30 | 0.30 |
| Ceteareth-12 | Nonionic emulsifier | 0.30 | 0.30 |
| Sodium myreth sulfate | Anionic surfactant | 1.96 | 1.96 |
| (C14 fatty alcohol, | | | |
| ethoxylated with | | | |
| 3 EO, sulfate, | | | |
| sodium salt) | | | |
| Sodium myreth sulfate | Anionic surfactant | 1.96 | 1.96 |
| (C14 fatty alcohol, | | | |
| ethoxylated with | | | |
| 3 EO, sulfate, | | | |
| sodium salt) | | | |
| Xanthan gum | Thickening polymer | 1.00 | 1.00 |
| Propylene glycol | Solvent | 2.00 | 2.00 |
| p-Toluylenediamine, | ODP | 0.111 | — |
| sulfate | | | |
| Resorcinol | ODP | 0.03 | — |
| m-Aminophenol | ODP | 0.002 | — |
| 4-Chlororesorcinol | ODP | 0.03 | — |
| 2-Methylresorcinol | ODP | 0.01 | — |
| Ammonium sulfate | Salt | 0.93 | — |
| (NH₄)₂SO₄ | | | |
| Sodium sulfite Na₂SO₃ | Salt | 0.40 | — |
| Vitamin C | Reducing agent | 0.10 | — |
| Dipicolinic acid | Stabilizer | — | 0.10 |
| Disodium pyrophosphate | Stabilizer | — | 0.91 |
| Hydroxyethane-1,1- | Stabilizer | 0.20 | 0.90 |
| diphosphonic acid | | | |
| Ammonia (NH3) | Alkalizing agent | 2.00 | — |
| Hydrogen peroxide | OX | — | 6.00 |
| Water | | To 100 | To 100 |
| Viscosity | | 20,000 | 20,000 |
| (22° C./Brookfield | | mPas | mPas |
| viscometer/spindle | | | |
| 5/4 rpm) | | | |

### Products and Abbreviations:

[1] Product W 37194: 1-Propanaminium, N,N,N-trimethyl-3-[(1-oxo-2-propenyl)amino]-, chloride, polymer with sodium 2-propenoate (INCI: ACRYLAMIDOPROPYLTRIMONIUM CHLORIDE/ACRYLATES COPOLYMER) (20% aqueous solution)
ODP: Oxidation dye precursors
OX: Oxidizing agent

### 2. Total Weight Ratio G1/G3

| | Preparation | Preparation |
|---|---|---|
| | (a) G1 | (b) G3 |
| Thickening polymers | 1.00 | 1.00 |
| Ratio G1/G3 | 1 | |

### 3. Total Weight Ratio G2/G4

| | Preparation | Preparation |
|---|---|---|
| | (a) G2 | (b) G4 |
| Fatty components | 5.1 | 5.1 |
| Ratio G2/G4 | 1 | |

### Example 2

The following preparations (a) and (b) were prepared (all quantities are given in % by weight of active substance):

| | | Prepa- | Prepa- |
|---|---|---|---|
| | | ration | ration |
| Ingredients | | (a) | (b) |
| Lanette D (cetearyl | Fatty component | 3.30 | 3.30 |
| alcohol) | | | |
| Lorol C12-C18 (techn.) | Fatty component | 1.20 | 1.20 |
| (C12-C18 fatty alcohols) | | | |
| Glyceryl oleate | Fatty component | 0.6 | 0.6 |
| Cocoglucosides | Nonionic emulsifier | 0.6 | 0.6 |
| Ceteareth-20 | Nonionic emulsifier | 0.30 | 0.30 |
| Ceteareth-12 | Nonionic emulsifier | 0.30 | 0.30 |
| Sodium acrylate, | Thickening polymer | 1.00 | 1.00 |
| (trimethyl- | | | |
| ammonio) | | | |
| propylacrylamide | | | |
| chloride, copolymer | | | |
| Sodium myreth sulfate | Anionic surfactant | 1.96 | 1.96 |
| (C14 fatty alcohol, | | | |
| ethoxylated with | | | |
| 3 EO, sulfate, | | | |
| sodium salt) | | | |
| Xanthan gum | Thickening polymer | 1.00 | 1.00 |
| Propylene glycol | Solvent | 2.00 | 2.00 |
| p-Toluylenediamine, | ODP | 0.111 | — |
| sulfate | | | |
| Resorcinol | ODP | 0.03 | — |
| m-Aminophenol | ODP | 0.002 | — |
| 4-Chlororesorcinol | ODP | 0.03 | — |
| 2-Methylresorcinol | ODP | 0.01 | — |
| Ammonium sulfate | Salt | 0.93 | — |
| (NH₄)₂SO₄ | | | |
| Sodium sulfite Na₂SO₃ | Salt | 0.40 | — |
| Vitamin C | Reducing agent | 0.10 | — |
| Dipicolinic acid | Stabilizer | — | 0.10 |
| Disodium pyrophosphate | Stabilizer | — | 0.91 |
| Hydroxyethane-1,1- | Stabilizer | 0.20 | 0.90 |
| diphosphonic acid | | | |
| Ammonia (NH3) | Alkalizing agent | 2.00 | — |
| Hydrogen peroxide | OX | — | 6.00 |
| Water | | To 100 | To 100 |
| Viscosity | | 20,000 | 20,000 |
| (22° C./Brookfield | | mPas | mPas |
| viscometer/spindle | | | |
| 5/4 rpm) | | | |

### Example 3

The following preparations (a) and (b) were prepared (all quantities are given in % by weight of active substance):

| | | Prepa- | Prepa- |
|---|---|---|---|
| | | ration | ration |
| Ingredients | | (a) | (b) |
| Lanette D (cetearyl alcohol) | Fatty component | 3.3 | 3.3 |
| Lorol C12-C18 (techn.) | Fatty component | 1.20 | 1.20 |
| (C12-C18 fatty alcohols) | | | |
| Ceteareth-20 | Nonionic emulsifier | 0.30 | 0.30 |
| Ceteareth-12 | Nonionic emulsifier | 0.30 | 0.30 |
| Cocoglucosides | Nonionic emulsifier | 1.00 | 1.00 |
| Sodium laureth-6-carboxylate | Anionic surfactant | 5.00 | 5.00 |
| Sodium myreth sulfate | Anionic surfactant | 0.98 | 0.98 |
| (C14 fatty alcohol, | | | |
| ethoxylated with | | | |
| 3 EO, sulfate, | | | |
| sodium salt) | | | |
| 1-(2-Hydroxyethyl)-4,5- | ODP | 1.90 | — |
| diaminopyrazole, sulfate | | | |
| m-Aminophenol | ODP | 0.3 | — |
| 2-Methylresorcinol | ODP | 0.05 | — |
| 5-Amino-2-methylphenol | ODP | 0.80 | — |
| Ammonium sulfate | Salt | 0.41 | — |
| (NH₄)₂SO₄ | | | |
| Sodium sulfite Na₂SO₃ | Salt | 0.40 | — |
| Vitamin C | Reducing agent | 0.10 | — |
| Potassium hydroxide | Alkalizing agent | 0.40 | 0.45 |
| Hydroxyethane-1,1- | Stabilizer | 0.2 | 1.5 |
| diphosphonic acid | | | |
| Propylene glycol | Solvent | 2.0 | 2.0 |
| Xanthan gum | Thickening polymer | 1.20 | 1.00 |
| Sodium acrylate, | Thickening polymer | 1.00 | 1.00 |
| (trimethyl- | | | |
| ammonio) | | | |
| propylacrylamide | | | |
| chloride, | | | |
| copolymer | | | |
| Ammonia (NH3) | Alkalizing agent | 1.63 | — |
| Dipicolinic acid | Stabilizer | — | 0.10 |
| Disodium pyrophosphate | Stabilizer | — | 0.03 |
| Hydrogen peroxide | OX | — | 6.10 |
| Water | | To 100 | To 100 |
| Viscosity | | 17,000 | 18,000 |
| (22° C./Brookfield | | | |
| viscometer/spindle | | | |
| 5/4 rpm) | | | |

### Example 4

The following preparations (a) and (b) were prepared (all quantities are given in % by weight of active substance):

| | | Prepa- | Prepa- |
|---|---|---|---|
| | | ration | ration |
| Ingredients | | (a) | (b) |
| Lanette D (cetearyl alcohol) | Fatty component | 4.95 | 4.95 |
| Lorol C12-C18 (techn.) | Fatty component | 1.80 | 1.80 |
| (C12-C 18 fatty alcohols) | | | |
| Ceteareth-20 | Nonionic emulsifier | 0.45 | 0.45 |
| Ceteareth-12 | Nonionic emulsifier | 0.45 | 0.45 |
| Cocoglucosides | Nonionic emulsifier | 1.00 | 1.00 |
| Sodium laureth-6- | Anionic surfactant | 5.00 | 5.00 |
| carboxylate | | | |
| Sodium myreth sulfate | Anionic surfactant | 0.98 | 0.98 |
| (C14 fatty alcohol, | | | |
| ethoxylated with | | | |
| 3 EO, sulfate, | | | |
| sodium salt) | | | |
| p-Toluylenediamine, | ODP | 2.20 | — |
| sulfate | | | |
| 2-Amino-4-[(2- | ODP | 0.05 | — |
| hydroxyethyl)amino]- | | | |
| anisole, sulfate | | | |
| Resorcinol | ODP | 0.80 | — |
| m-Aminophenol | ODP | 0.30 | — |
| Sodium sulfite Na₂SO₃ | Salt | 0.40 | — |
| Vitamin C | Reducing agent | 0.10 | — |
| Hydroxyethane-1,1- | Stabilizer | 0.20 | 1.50 |
| diphosphonic acid | | | |
| Propylene glycol | Solvent | 3.00 | 3.00 |
| Xanthan gum | Thickening polymer | 1.50 | 1.50 |
| Sodium acrylate, | Thickening polymer | 1.00 | 1.00 |
| (trimethylammonio)- | | | |
| propylacrylamide | | | |
| chloride, | | | |
| copolymer | | | |
| Ammonia (NH3) | Alkalizing agent | 1.63 | — |
| Dipicolinic acid | Stabilizer | — | 0.1 |
| Disodium pyrophosphate | Stabilizer | — | 0.03 |
| Potassium hydroxide | Alkalizing agent | 1.80 | 0.91 |
| Hydrogen peroxide | OX | — | 6.1 |
| Water | | To 100 | To 100 |
| Viscosity | | 42,3000 | 43,000 |
| (22° C./Brookfield | | | |
| viscometer/spindle | | | |
| 5/4 rpm) | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A product for the oxidative coloring and/or lightening of keratinic fibers, including
(1) a dispenser, which
has two chambers (A) and (B) separate from one another, and
has an outlet opening (C), which is in communication with chamber (A) and with chamber (B),
(2) a preparation (a) in chamber (A), including in a cosmetic carrier
one or more thickening polymers in a total amount by weight (G1) of 0.1 to 20% by weight, based on the total weight of preparation (a),
one or more fatty components in a total amount by weight (G2) of 0.1 to 20% by weight, based on the total weight of preparation (a),
(3) a preparation (b) in chamber (B), including in a cosmetic carrier
one or more thickening polymers in a total amount by weight (G3) of 0.1 to 20% by weight, based on the total weight of preparation (b),
one or more fatty components in a total amount by weight (G4) of 0.1 to 20% by weight, based on the total weight of preparation (b),
wherein
the weight ratio of G1/G3 has a value of 0.5 to 2.0 and
the weight ratio of G2/G4 has a value of 0.5 to 2.0.

2. The product according to claim 1, wherein the weight ratio of G1/G3 has a value of 0.6 to 1.8.

3. The product according to claim 1, wherein the weight ratio of G2/G4 has a value of 0.6 to 1.8.

4. The product according to claim 1, wherein the weight ratio (G1+G2)/(G3+G4) has a value of 0.3 to 3.0.

5. The product according to claim 1, wherein
preparation (a) includes as thickening polymer(s) one or more polymers from the group consisting of polysaccharides, acrylic acid polymers, acrylic acid copolymers, methacrylic acid polymers, and methacrylic acid copolymers, and
preparation (b) includes as thickening polymer(s) one or more polymers from the group consisting of polysaccharides, acrylic acid polymers, acrylic acid copolymers, methacrylic acid polymers, and methacrylic acid copolymers.

6. The product according to claim 1, wherein
preparation (a) includes as thickening polymer(s) one or more polysaccharides from the group consisting of carboxymethylcelluloses, alginic acids, xanthan gum, and the physiologically acceptable salts thereof, and
preparation (b) includes as thickening polymer(s) one or more polysaccharides from the group consisting of carboxymethylcelluloses, alginic acids, xanthan gum, and the physiologically acceptable salts thereof.

7. The product according to claim 1, wherein preparation (a) and preparation (b) include the same thickening polymers.

8. The product according to claim 1, wherein
preparation (a) includes the thickening polymer(s) in a total amount by weight (G1) of 0.5 to 9.5% by weight based on the total weight of preparation (a), and
preparation (b) includes the thickening polymer(s) in a total amount by weight (G3) of 0.5 to 9.5% by weight based on the total weight of preparation (b).

9. The product according to claim 1, wherein
preparation (a) includes as the fatty component(s) one or more compounds from the group consisting of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and hydrocarbons, and
preparation (b) includes as the fatty component(s) one or more compounds from the group consisting of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and hydrocarbons.

10. The product according to claim 1, wherein
preparation (a) includes the fatty component(s) in a total amount by weight (G2) of 0.25 to 9.5% by weight based on the total weight of preparation (a), and
preparation (b) includes the fatty component(s) in a total amount by weight (G4) of 0.25 to 9.5% by weight based on the total weight of preparation (a).

11. The product according to claim 1, wherein
preparation (a) has a viscosity of 1000 to 100,000 mPas at 22° C. using a Brookfield viscometer/spindle 5/4 rpm,
preparation (b) has a viscosity of 1000 to 100,000 mPas at 22° C. using a Brookfield viscometer/spindle 5/4 rpm, and
the ratio of the viscosities V1/V2 has a value of 0.3 to 3.0.

12. The product according to claim 1, wherein
the dispenser is an aerosol pressurized container,
outlet opening (C) is an aerosol dispensing device, and
preparations (a) and (b) in each case further include at least one propellant from the group consisting of propane, propene, n-butane, isobutane, isobutene, n-pentane, pentene, isopentane, isopentene, air, nitrogen, argon, N₂O, and/or CO₂.

13. The product according claim 1, wherein
the dispenser is a dispenser for pasty materials, a tube, or a squeeze tube, and
outlet opening (C) is a pump element, a valve, or a dispensing device for pasty materials.

14. A method for the coloring and/or lightening of keratinic fibers with the use of a product according to claim 1, including
emptying the dispenser continuously in one step or in batches in a plurality of steps, such that in each step, constant amounts of preparations (a) and (b) are removed each time from chambers (A) and (B) via common outlet opening (C) of the dispenser until both chambers (A) and (B) have been emptied completely.

15. The method according to claim 14, wherein
in each step the same amounts of preparations (a) and (b) are removed each time from chambers (A) and (B) via common outlet opening (C) of the dispenser until both chambers (A) and (B) have been emptied completely.
